(19) United States
(12) Patent Application Publication
(10) Pub. No.: US 2024/0042449 A1
(43) Pub. Date: Feb. 8, 2024

(54) TEST DEVICE, TEST METHOD AND MANUFACTURING METHOD FOR TEST DEVICE FOR CARRYING OUT A TEST DIRECTED TO THE DETECTION OF AN ORGANIC STRUCTURE

(71) Applicant: Solios Diagnostics GmbH, Tuebingen (DE)

(72) Inventor: Dieter Hassberg, Tuebingen (DE)

(21) Appl. No.: 18/382,747

(22) Filed: Oct. 23, 2023

Related U.S. Application Data
(63) Continuation of application No. PCT/EP22/60305, filed on Apr. 19, 2022.

(30) Foreign Application Priority Data
Apr. 23, 2021 (DE) 10 2021 110 457.2

(51) Int. Cl. B01L 3/00

(52) U.S. Cl. CPC B01L 3/523; B01L 2300/042; B01L 2300/069; B01L 2300/0832

(57) ABSTRACT

The present invention relates to a test device and a test method for carrying out a test directed to the detection of an organic structure. The test device comprises a housing wherein a receiving area for receiving a sample collection stick, a liquid reservoir forming a separated area within the housing and filled with a reaction solution, and an analysis area are received. Preferably, the reaction solution is received in a cartridge sealed at both ends by a separation layer.

### CROSS-REFERENCE TO RELATED APPLICATIONS

This application is a continuation of copending international patent application PCT/EP2022/060305 filed on 19 Apr. 2022 and designating the U.S., which has been published in German, and claims priority from German patent application DE 10 2021 110 457.2 filed on 23 Apr. 2021. The entire contents of these prior applications are incorporated herein by reference.

### FIELD OF THE INVENTION

The present invention relates to a test device and method for carrying out a test directed to the detection of an organic structure. The invention further relates to a manufacturing method for manufacturing such a test device.

### BACKGROUND OF THE INVENTION

A test directed to the detection of an organic structure is carried out, for example, to detect infection with a virus in a subject. The organic structure in this case can be a surface structure of the virus such as an antigen. Due to the worldwide increase in infectious diseases, early detection by means of simple and safe tests is of great importance. In addition, a rapid test result is usually desired in order to obtain information on a daily or even hourly basis as to whether an infection is present in the test person. Against this background, it is desirable to have a possibility to perform tests on a large number of test persons in a particularly simple and fast way. In addition, it is desirable to avoid, as far as possible, incorrect carrying out of the tests due to improper use.

Methods, test devices and test kits for carrying out a test directed to the detection of an organic structure are known from the prior art. For example, Chinese utility model application CN 211148669 U describes a test kit with a test cassette and a test method. The test is carried out in several individual steps. Here, a sample diluent is first added to a test tube. Then a cotton swab or swab stick with a sample is dipped into the sample diluent. Stirring or pressing the stick against the wall of the sample tube causes the sample to transfer into the sample diluent. The stick is then pulled-out of the sample tube and a filter cap is applied to the sample tube. Then two drops of the sample solution with about 100 μl are applied to a sample opening of a test cassette. Finally, the sample solution is analyzed in the test cassette.

The prior art method just described by way of example using utility model application CN 211148669 U is in need of improvement in that it comprises many individual steps and is therefore complicated and time-consuming. The stirring necessary to prepare the sample solution and the pressing of the stick against the wall of the test tube and the dosing of the sample solution at the opening of the test cassette also involve the risk of improper performance, in particular by medically untrained personnel. Furthermore, the test kit requires a total of at least four parts, namely the cotton swab or swab stick, the sample tube, the filter cap, the test cassette, and usually an additional vial containing the sample diluent. In addition, a sufficiently sterile base, such as a table, is ideally required to place the parts of the test kit that are not currently required. This makes the test kit and the test device, which also consists of several individual parts, more difficult to handle.

From U.S. Pat. No. 4,770,853 A, a test device for an antigen test is known, in which a sample collection stick is held on a cover and recessed in the housing before use. After sampling, the cover is unscrewed, and the reaction solution is brought into contact with the sample after piercing a membrane. By further movement of the sample collection stick, a second membrane is pierced and the sample solution passes through an opening to an analysis area applied to the outside.

From U.S. Pat. No. 5,965,453 A, a further test device for an antigen test is known, in which a sample collection stick is held on a cover before use and is recessed in the housing. After sampling, the cover is unscrewed, and the reaction solution is brought into contact with the sample after piercing a membrane. Finally, the sample liquid is fed to an analysis area at the bottom of the housing. For analysis, the entire device or the lower end with the sample solution can be placed in a luminometer to determine the color and activate a counter.

### SUMMARY OF THE INVENTION

Against this background, the present invention has the task of providing a test device and a test method for carrying out a test directed to the detection of an organic structure, which enable the tests to be carried out rapidly and more reliably and simply than in the prior art.

According to the invention, this problem is solved by a test device for carrying out a test directed to the detection of an organic structure, with a housing comprising:

a receiving area for receiving a sample collection stick;

a liquid reservoir forming a separated area within the housing and filled with a reaction solution; and

an analysis area integrated into the housing.

According to a further embodiment of the invention, a method for carrying out a test directed to the detection of an organic structure is disclosed, in particular using a test device of the type mentioned above, with the following steps:

inserting a sample collection stick, on which at least one sample is located, into a receiving area of a housing of a test device;

moving the sample collection stick into a liquid reservoir that forms a separate area within the housing and is filled with a reaction solution so that the at least one sample contacts the reaction solution, thereby forming a sample solution; and

moving the sample collection stick so that at least portion of the sample solution reaches an analysis area of the housing.

According to the invention, all components of the test device are received in the housing. This ensures a particularly compact design and simple packaging and handling. Thus, no additional, external components are required. Consequently, the number of individual components of the test device and thus the complexity can be reduced compared to the prior art, making the test device according to the invention particularly easy to handle.

The housing can be configured as a one-piece component.

In the context of the present invention, a one-piece component of the housing is understood to be a component whose individual areas and/or sections and/or parts are firmly connected to one another such that a unit in the sense of a single piece is formed. For example, areas and/or sections and/or parts of the component for this purpose are non-detachably connected to one another, in particular in a materially bonded manner, so that they form an integral unit. Alternatively or additionally, areas and/or sections and/or parts of the housing can also be welded, bonded, injection-molded, screwed and/or plugged together and/or fixed to one another in other suitable ways to form a unit in the sense of a one-piece component.

The housing or one-piece component serves to accommodate all components required to carry out the test as an integral unit. If the one-piece component is manufactured by connecting a plurality of subcomponents using a connection method, such as heat sealing, welding, or bonding, the manufacturing and assembly is facilitated.

In a further embodiment of the invention, the term “housing” is to be replaced by the term “one-piece component” in each case in certain embodiments, wherein the term “one-piece component” is to be understood in the manner explained above.

The invention comprises inserting a sample collection stick having at least one sample thereon into a receiving area of a housing of a test device. It is understood herein that the sample collection stick and the housing are moved relative to each other such that the sample collection stick is inserted into the receiving area of the housing. Thereby, on the one hand, only the sample collection stick can be moved and the housing can remain in a fixed position. On the other hand, only the housing can be moved and the sample collection stick can remain at a fixed position. Finally, both the sample collection stick and the housing can be moved and neither the sample collection stick nor the housing remain in a fixed position. In either case, the movement of the sample collection stick and/or the housing relative to each other is such that the sample collection stick is inserted into the receiving area of the housing. Insertion of the sample collection stick into the receiving area of the housing causes the sample located on the sample collection stick to be provided within the housing for carrying out the test.

In accordance with a further embodiment of the invention, an extraction area is further provided within the housing.

By an extraction area is meant any area within the housing that enables at least a portion of the sample solution to enter the analysis area of the housing. In this regard, a concentration of the sample solution can be achieved by a particular design of the extraction area. The sample solution thus enters the analysis area, where the actual analysis and thus the performance of the test can take place.

The extraction area can be configured as conical, funnel-shaped, or plunger-shaped (similar to a syringe head) and can comprise an opening such that when the sample collection stick is moved into the extraction area, at least a portion of the sample solution passes through the opening of the conically shaped extraction area and into the analysis region. Such a shaped component can also be provided, configured as a separate device and possibly received in the fluid reservoir. The shape and the opening of the extraction area, when the sample collection stick is pressed against the wall of the extraction area, cause a process-safe supply of the sample solution to the analysis area as well as a precise dosage of which amount of sample solution is fed into the analysis area. Alternatively, the extraction area can be configured as a cross-sectional taper.

In accordance with a further preferred embodiment of the invention, the extraction area comprises the shape of a planar and/or curved surface with an opening, wherein when the sample collection stick is moved into the extraction area, at least some of the sample solution passes through the opening of the planar and/or curved surface of the extraction area into the analysis area. For example, the extraction area is configured in a conical or plunger-shape (similar to a syringe head). The shape of a flat and/or curved surface as well as the opening of the extraction area also cause a process-safe supply of the sample solution into the analysis area as well as a precise dosing of the amount of sample solution fed into the analysis area when the sample collection stick is pressed against the surface of the extraction area.

In accordance with a further embodiment of the invention, the housing receives a cartridge containing the fluid reservoir, which is delimited at both ends by separating layers, in particular membranes.

By using a cartridge in which the reaction solution is contained and delimited at both ends by a separating layer, in particular in the form of a membrane, the provision of the reaction solution is decoupled from the provision of the other components of the test device. In this way, manufacture and assembly are simplified. Furthermore, precise dosing of the reaction solution is facilitated and the risk of contamination is reduced.

According to a further embodiment of the invention, the cartridge is fixed in the housing, preferably by means of a clamp or click connection.

This simplifies the assembly.

In accordance with a further embodiment of the invention, the extraction area is provided at the outer end of the cartridge.

According to an alternative embodiment of the invention, the extraction area is received within the cartridge.

In some embodiments of the invention, an extraction area is omitted so that the reaction solution from the liquid reservoir immediately enters the analysis area when the interface is punctured.

In accordance with a further embodiment of the invention, a stripping element is provided within the cartridge for stripping the sample collection stick.

This assists in transferring the sample material from the sample collection stick to the reaction solution.

In accordance with a further embodiment of the invention, a method is disclosed wherein the at least one sample located on the sample collection stick is a sample collected by a nasopharyngeal swab, a sample collected by a nasal swab, a sample collected by a throat swab, and/or a saliva sample. It can also be another secretion (e.g., a wound secretion or tear fluid) or, for example, a stool sample.

According to the invention, the fluid reservoir is delimited by at least one separation layer that is at least partially broken by moving the sample collection stick against the separation layer so that the sample comes into contact with the reaction solution.

The method according to the invention further comprises moving the sample collection stick into a liquid reservoir forming a separated area within the housing and filled with a reaction solution, such that the at least one sample contacts the reaction solution, thereby forming a sample solution. Moving the sample collection stick into the fluid reservoir can also comprise initially moving the sample collection stick toward the fluid reservoir, wherein the sample collection stick is moved toward the fluid reservoir until the sample collection stick abuts the fluid reservoir. During this movement or subsequently, the sample collection stick can be pressed against the liquid reservoir. Thereby the pressing is also to be understood reciprocally. This means that pressing the sample collection stick against the liquid reservoir can equally be pressing the liquid reservoir against the sample collection stick. Furthermore, the moving is also to be understood relatively. This means that moving the sample collection stick towards the liquid reservoir can also be moving the liquid reservoir towards the sample collection stick or moving both the sample collection stick and the liquid reservoir towards each other.

According to the invention, the liquid reservoir located within the housing is filled with a reaction solution. Upon contact with the sample located on the sample collection stick, the reaction solution forms, together with the sample, a sample solution suitable for analysis in the analysis area of the housing. In particular, this can comprise that the sample is diluted by the reaction solution and/or dissolves in the reaction solution so that a sample solution is formed in this way. Furthermore, in some embodiments of the method according to the invention, a physical and/or chemical and/or biochemical process and/or reaction can also take place, involving both components of the sample and components of the reaction solution. This process can take place both immediately upon contact of the sample with the reaction solution and after the at least one sample has been dissolved in the reaction solution. For example, the reaction solution is an extraction buffer solution by which lipid components of the sample are dissolved.

Moving the sample collection stick into the fluid reservoir results in the at least one sample coming into contact with the reaction solution, thereby causing a sample solution to be formed that is suitable for analysis in the analysis region of the housing, thereby enabling the test to be performed.

The fluid reservoir can overlap (partially or completely) with or merge into the extraction area. Moving the sample collection stick into the extraction area can also include initially moving the sample collection stick toward the extraction area, wherein the sample collection stick is moved toward the extraction area until the sample collection stick abuts the extraction area. In the course of this movement or subsequently, the sample collection stick can be pressed against the extraction area (i.e. against a resistance in the extraction area). Thereby, the pressing is also to be understood reciprocally. This means that pressing the sample collection stick against the extraction area can equally be pressing the extraction area against the sample collection stick. Furthermore, the moving is also to be understood relatively. This means that moving the sample collection stick in the direction of the extraction area can also be moving the extraction area in the direction of the sample collection stick or moving both the sample collection stick and the extraction area towards each other.

According to the invention, with the movement of the sample collection stick towards the analysis area or into the extraction area of the housing, at least part of the sample solution enters the analysis area of the housing. In particular, this can occur under the influence of a pressure or force. For example, the sample solution can be forced into the analysis area as a result of pressing the sample collection stick against a resistance (such as a wall) in the extraction area. Alternatively or additionally, the sample solution can flow into the analysis area under the influence of gravity, by capillary action, or by some other force acting on the sample solution, for example. In any case, moving the sample collection stick into the extraction area of the housing results in at least some of the sample solution entering the analysis area of the housing, thereby enabling the sample solution to be analyzed in the analysis area and thus enabling the test to be performed.

According to the invention, error-prone and time-consuming method steps of the prior art, such as filling a sample diluent into a sample tube, applying a filter cap to the sample tube, and applying a defined amount of the sample solution to a sample opening of a test cassette, are not required. Consequently, the complexity and error-proneness of the method according to the invention can be reduced compared to the prior art and, moreover, time can be saved. Moreover, the steps of the method according to the invention just mentioned are all based on the movement or force action of the sample collection stick relative to the housing of the test device, whereby the method can also be performed in a particularly simple and at the same time application-safe manner.

According to a preferred embodiment of the method of the invention, the at least one sample located on the sample collection stick is a sample collected by a nasopharyngeal swab, a sample collected by a nasal swab, a sample collected by a pharyngeal swab, and/or a saliva sample. Samples from the nasal and/or pharyngeal region or saliva of the subject are often used to perform tests directed at detecting an organic structure in the subject. Such samples can be analyzed with the method according to the invention particularly quickly, easily and at the same time reliably.

In accordance with a further embodiment of the invention, a test device is disclosed wherein the analysis section comprises:

an analysis element;

for analyzing a sample solution, in particular a test strip according to the lateral flow principle, or an electronic or chip-based analysis element; and/or

a window for reading a test result of the analysis element.

According to a further embodiment of the invention, the receiving area, the liquid reservoir, the analysis area, and, if present, the extraction area are arranged one behind the other in a longitudinal direction of the housing.

This ensures a test device that is particularly easy to handle and read.

In accordance with a further embodiment of the invention, a test device is disclosed in which the housing comprises a laterally outwardly projecting area in which the analysis element is received.

In this way, the overall length of the test device can be shortened compared to the aforementioned embodiment and a more compact design can be ensured.

According to a further embodiment of the invention, the extraction area is received in the cartridge.

In accordance with a further embodiment of the invention, a cover is provided which is removably fixed on one end of the housing.

In this regard, the cover can be retained on the end of the housing by means of a screw mechanism, a click mechanism, or a combined screw-and-click mechanism.

According to a further embodiment of the invention, the cover is applied to an opening of the housing by means of a two-stage closure mechanism, in particular by means of a two-stage click mechanism or by means of a two-stage screw mechanism or by means of a two-stage combined click-screw or screw-click mechanism.

In this way, in the first stage, the sample can first be brought into contact with the reaction solution by a movement of the cover, and in the second stage, the sample solution can be dispensed toward the analysis area.

Preferably, the sample collection stick is held in the housing and secured by the cover.

Thereby, the sample collection stick can be fixed to the cover or detachably fixed thereto. Thus, the sample collection stick can be held directly on the cover and handled therewith.

In accordance with a further embodiment of the invention, the sample collection stick is movable by a movement of the cover into the housing against a first separation layer of the liquid reservoir for opening thereof, and movable against the second separation layer for releasing the liquid of the liquid reservoir towards the analysis area.

According to a further preferred embodiment of the method according to the invention, the direction of movement (or force) of the sample collection stick into the liquid reservoir and the direction of movement (or force) of the sample collection stick into the extraction region are substantially identical. By this is meant that the angle between the two directions of movement or force is not more than +/−30°, in particular not more than +/−15°. Preferably, the directions are identical. In particular, the direction of the movement with which the insertion of the sample collection stick into the receiving area of the housing takes place and the directions of the two said movements are also substantially identical. However, the angle between the direction of the movements can also be more than +/−30° in each case. For example, the steps of the method according to the invention mentioned at the beginning can be carried out by means of a continuous movement with substantially identical direction. By the substantially identical direction of force and/or movement, the method according to the invention can be performed in a particularly simple, fast and application-safe manner, since only the sample collection stick has to be successively pushed along a direction relative to the housing of the test device.

According to a further preferred embodiment of the invention, moving the sample collection stick into the fluid reservoir and/or moving the sample collection stick into the extraction area is performed by applying a cover to an opening of the housing. In particular, this is the opening by which the sample collection stick was inserted into the housing. Through this, it is possible to precisely adjust the respective pressure forces acting on the liquid reservoir and/or the extraction area of the housing and/or the depth of insertion into the housing via the locking mechanism of the cover (such as a screw thread), so that too high or too low pressure forces can be avoided. Consequently, the risk of improper performance of the method, in particular by medically untrained personnel, is reduced, thus improving application safety.

According to a further preferred embodiment of the invention, the application of the cover is performed in two stages, wherein in the first stage the movement of the sample collection stick into the liquid reservoir is performed and in the second stage the movement of the sample collection stick into the extraction area is performed. This enables the contact between the at least one sample with the reaction solution and the supply of the sample solution to the analysis area to take place separately in time, so that the flexibility of the method according to the invention is further improved.

In accordance with a further preferred embodiment of the invention, the cover comprises a fixation element for at least temporarily fixing a sample collection stick. The fixation element can be, for example, a hollow cylinder adapted to a diameter of the sample collection stick and open on one side. The fixing element prevents the sample collection stick from sliding off when the sample collection stick is pressed against the liquid reservoir and/or the extraction area by means of the cover and thus enables a particularly process-safe performance of a corresponding preferred embodiment of the method according to the invention.

In accordance with a further preferred embodiment of the invention, a waiting time of between 0.1 s and 3600 s, in particular between 1 s and 600 s, is observed between moving the sample collection stick into the liquid reservoir and moving the sample collection stick into the extraction area. This can ensure that the at least one sample can dissolve in the reaction solution, as well as that the physical and/or chemical and/or biochemical processes and/or chemical and/or biochemical reactions that may take place can proceed before the sample solution is fed to the analysis area. However, the observance of a waiting time is not mandatory for all tests directed to the detection of an organic structure.

In accordance with a further embodiment of the invention, the invention further comprises reading a test result by a window of the analysis area of the housing. The test result can comprise, for example, the test element in the analysis area of the housing taking on a particular color, at least one strip appearing on the test element, or the test element displaying information about the test result in some other suitable way. By reading the test result through a window, a process-safe evaluation of the test is enabled, if necessary also by medically untrained personnel.

According to a further embodiment of the invention, the method is a method for carrying out a rapid test directed to the detection of an organic structure. By a rapid test is meant a test which provides a test result within a period of less than 120 minutes, in particular less than 30 minutes. In particular, this can be a method for carrying out a test directed to the detection of SARS-CoV-2 or an antigen of SARS-CoV-2.

According to a further embodiment of the invention, the at least one separation layer of the liquid reservoir is designed to be penetrated by a sample collection stick inserted into the housing and pressed against the at least one separation layer, so that a sample located on the sample collection stick comes into contact with the reaction solution. For example, the at least one separation layer can have a strength that is just high enough for the separation layer to be penetrated by the compressive forces that usually act on the separation layer when the sample collection stick is pressed against the separating layer. Alternatively or additionally, the at least one separating layer can also have a predetermined penetration point, for example. Because the at least one separation layer is designed to be penetrated by a sample collection stick, the tests can be carried out in a simple and safe manner, even by untrained medical personnel.

According to a further embodiment of the invention, the analysis area comprises a window for reading a test result of the analysis element. The window can either be open to the outside or can comprise a transparent cover. As previously explained, this enables a process-safe evaluation of the test, if necessary also by medically untrained personnel.

According to a further embodiment of the invention, the two-stage locking mechanism is a two-stage click mechanism, an (at least) two-stage screw mechanism (for example with different thread pitches and/or interrupted thread) or an (at least) two-stage combined click-screw or screw-click mechanism. Thereby, any combinations between a screw mechanism and/or a click mechanism for the two-stage locking mechanism are conceivable. The provision of a screw mechanism enables the desired movements to be mapped and/or the respective pressure forces with which the pressing of the sample collection stick into the liquid reservoir and/or the pressing of the sample collection stick into the extraction area to be precisely adjusted, so that the application safety of the test device is improved. On the other hand, the provision of a click mechanism enables a quick application of the cover. Both a screw mechanism and a click mechanism also enable the cover to be applied to the housing by hand.

According to a further embodiment of the invention, an idle time is provided between the two stages of the two-stage locking mechanism. If, in this case, as explained upwardly, in the first stage the movement of the sample collection stick into the liquid reservoir and in the second stage the movement of the sample collection stick into the extraction area takes place, then, when performing a method according to the invention, the contact between the at least one sample with the reaction solution and the supply of the sample solution into the analysis area can take place separately in time, so that an improvement in the flexibility in performing the test can be achieved. In addition, the test device enables a waiting time to be observed, if necessary, before the sample solution is fed to the analysis area.

In accordance with a further embodiment of the invention, the cover comprises a fixation element for at least temporarily fixing a sample collection stick. The fixation element can be, for example, a hollow cylinder adapted to a diameter of the sample collection stick and open on one side. As already explained in connection with the method according to the invention, a fixation element enables a particularly process-safe performance of a corresponding preferred embodiment of the method according to the invention.

According to a further embodiment of the invention, the test device, in particular the housing of the test device, is configured as a hand-held device. In particular, the housing thereby comprises the shape of a tube or pipe. In this context, a hand-held device is understood to be a device that is held in the hand during its use. In particular, the test device is held in the hand when at least one step of the method according to the invention is performed with it. This means that a sterile base, such as a table, is not required to support the test device when performing the test. The test device according to the invention is thus characterized by particularly good manageability.

In accordance with a further embodiment of the invention, the housing is composed of at least two housing elements which are connected to one another in a sealing manner by a material connection, wherein the analysis element and the cartridge are fixed between the housing elements in their intended position.

In this way, particularly simple manufacture and assembly in large quantities are ensured.

To the extent that an extraction area and/or stripping element is provided and is not configured as an integral part of the housing, this element can be received between the two housing members.

In accordance with the invention, there is further disclosed a method of manufacturing a test device for carrying out a test directed to the detection of an organic structure, comprising a housing made of plastic material and comprising a receiving area for receiving a sample collection stick, and a liquid reservoir forming a separated area inside the housing and filled with a reaction solution, and an analysis area integrated into the housing, wherein the housing is composed of at least two housing elements in the longitudinal direction, with the following steps:

manufacturing a first housing element;

manufacturing a second housing member;

providing a sample collection stick;

manufacturing a cover of plastic material longitudinally fixable to the housing with a screw or click connection; and fixing the sample collection stick to the cover;

manufacturing a cartridge containing at least one liquid reservoir, wherein each liquid reservoir is delimited by separation layers, in particular membranes;

providing an analysis element;

wherein the housing is divided in longitudinal direction into at least the first and the second housing element such that the cartridge and the analysis element are insertable into areas of the first housing element provided for this purpose;

inserting the cartridge and the analysis element into the first housing element;

placing the second housing element onto the first housing element;

connecting the first and second housing elements in a material-locking manner to form the housing; and

attaching the cover with the sample collection stick held thereto to the screw or click connection of the housing; and inserting the sample collection stick into a cavity of the housing to such an extent that an end of the sampling rod intended for sampling is held to the cartridge with a distance to a first interface.

In this way, simple and reliable production in large quantities is enabled.

The material-locking connection can be made by welding, in particular ultrasonic welding, heat sealing or gluing.

Insofar as an extraction area is provided as a separate component, this can also be inserted with the first housing element before the second housing element is placed thereon. Alternatively, an extraction area can be configured with the first and second housing elements as an integral part of the housing.

Within the scope of the present disclosure, all features disclosed in connection with the method according to the invention are also to be understood as equally disclosed (in particular as means for performing corresponding method steps) for the test device according to the invention. Conversely, all features which are disclosed only in connection with the test device according to the invention are also to be understood as disclosed (in particular as corresponding method steps) for the method according to the invention.

According to the invention, the test device comprises a sample collection stick adapted to the test device. In particular, the sample collection stick is adapted to a length of the housing. For example, the sample collection stick can be adapted to a length of the housing in that the sample collection stick is moved into the fluid reservoir of the housing when a cover is applied to the opening of the housing by which the sample collection stick was inserted into the housing. Further, the sample collection stick can be adapted to a length of the housing in that the sample collection stick is moved into the extraction region of the housing when a cover is moved between the first and second stages of a two-stage closure mechanism of the housing. An adaptation of the sample collection stick to the test device has the effect that the method according to the invention can be performed with the test kit according to the invention in a particularly application-safe manner.

According to a preferred embodiment of the test kit of the invention, the sample collection stick comprises a compressible, open-pored sample receiving area. The sample receiving area can be configured, for example, as a cotton ball or sponge. An embodiment as a flocked head or a head provided with hair has proved to be particularly advantageous. If it is a stool sample, a spoon-like design is preferred.

In this way, a particularly effective sampling and sample transfer to the reaction solution is ensured.

After contact with the reaction solution, the sample receiving area of the sample collection stick is soaked or filled with the sample solution. If the sample collection stick is now moved into the extraction area of the housing, the sample receiving area can be compressed, for example, so that at least part of the sample solution escapes from the pores of the sample receiving area and can enter the analysis area of the housing. The supply of the sample solution to the analysis area can thereby occur as a result of the pressure resulting from pushing the sample collection stick into the extraction area. Alternatively or additionally, the supply of the sample solution to the analysis area can also take place under the influence of the gravity force or another force acting on the sample solution.

It is understood that the above features and those to be explained below are usable not only in the combination indicated, but also in other combinations or alone, without departing from the scope of the invention. Further features and advantages of the invention will be apparent from the following description with reference to the drawings. The figures are not to scale and are intended merely to reflect the general concept of the present invention by way of example. In particular, features included in the figures are in no way intended to be considered a necessary part of the present invention.

### BRIEF DESCRIPTION OF THE DRAWINGS

FIG. 1 shows a schematic flow diagram of an exemplary embodiment of a method according to the invention;

FIG. 2 shows a schematic illustration of a test kit according to the invention with a first exemplary embodiment of a test device according to the invention and a sample collection stick adapted to the test device;

FIG. 3a shows a schematic representation of the test kit of FIG. 2 in an arrangement corresponding to a step of the method according to the invention;

FIG. 3b shows a schematic representation of the test kit from FIG. 2 in an arrangement which corresponds to a further step of the method according to the invention;

FIG. 4 shows a schematic representation of a test kit according to the invention with a second exemplary embodiment of a test device according to the invention and a sample collection stick adapted to the test device;

FIG. 5a shows a schematic representation of the test kit of FIG. 4 in an arrangement corresponding to a step of the method according to the invention;

FIG. 5b shows a schematic representation of the test kit from FIG. 4 in an arrangement corresponding to a further step of the method according to the invention;

FIG. 6a shows a longitudinal view of a further embodiment of a test device according to the invention;

FIG. 6b shows a longitudinal view of the test device according to FIG. 6a, but in a representation rotated by 90°;

FIG. 6c shows a longitudinal section through the test device according to FIG. 6b along line VI-VI;

FIG. 7a-c show illustrations of the test device according to FIG. 6a-c, but of a movement of the sample collection stick into an extraction area for squeezing out the sample liquid into the analysis area;

FIG. 8 shows a partial section through a slightly modified version of the test device according to FIG. 6 only in the area of the cartridge and the extraction area;

FIG. 9 shows a partial section through a further modified version of the test device according to FIG. 6 only in the area of the cartridge and the extraction area;

FIG. 10 shows a top view of a stripping element located in the cartridge according to FIG. 8;

FIG. 11 shows a partial section through a further modified version of the test device according to FIG. 6 only in the area of the cartridge, without extraction area and without stripping element and

FIG. 12 shows a partial section through a further modified version of the test device according to FIG. 6 only in the area of the cartridge, with extraction area, but without stripping element.

### EXAMPLES

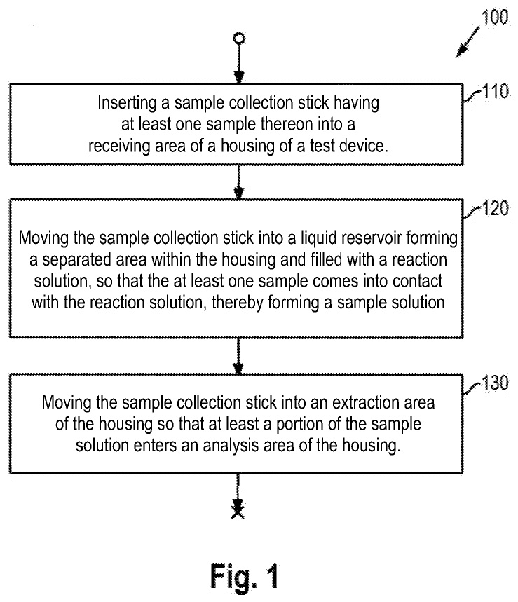

The method is described below by way of example with reference to FIG. 1. FIG. 1 shows a schematic flow diagram of an exemplary embodiment of the method according to the invention.

In this embodiment, the method 100 comprises inserting 110 a sample collection stick having at least one sample thereon into a receiving area of a housing of a test device. Further, the method comprises moving and pushing 120 the sample collection stick into and against a fluid reservoir forming a separated area within the housing and filled with a reaction solution such that the at least one sample contacts the reaction solution, thereby forming a sample solution. Finally, the method comprises moving and pushing 130 the sample collection stick into and against an extraction area of the housing such that at least a portion of the sample solution enters an analysis area of the housing.

Although the individual method steps in FIG. 1 are illustrated in a specific sequence, any way of carrying out the method 100 that makes sense to a skilled person is conceivable, even if this results in a different sequence or no sequence at all, i.e. simultaneous execution of individual or all method steps. In the method 100 shown in FIG. 1, error-prone and time-consuming method steps of the prior art, such as filling a sample diluent into a sample tube, applying a filter cap to the sample tube, and applying a defined amount of the sample solution to a sample opening of a test cassette, are not necessary, so that the complexity and error-proneness of the method can be reduced compared to the prior art, and time can also be saved.

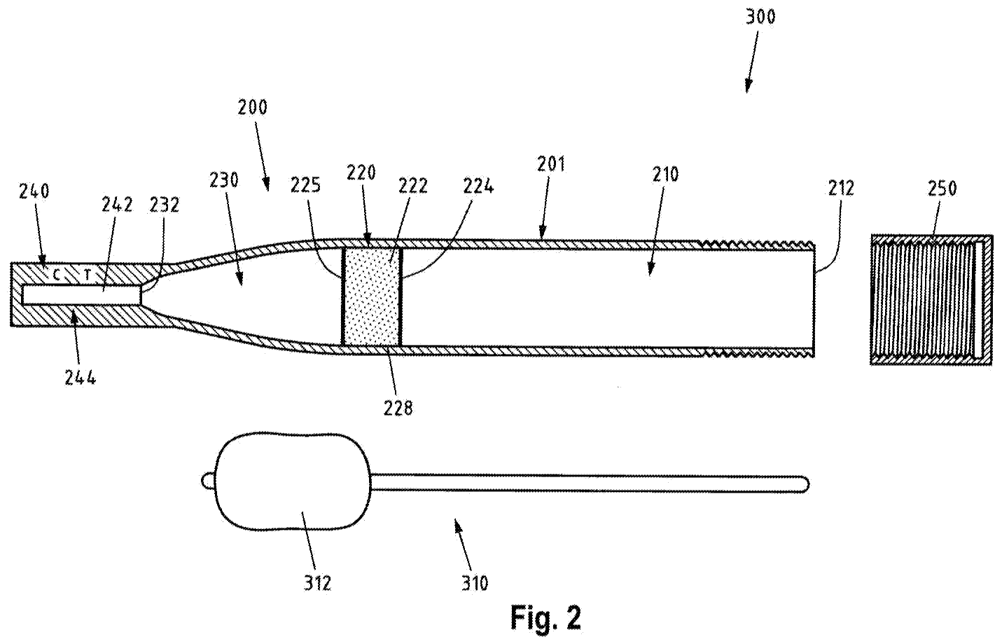

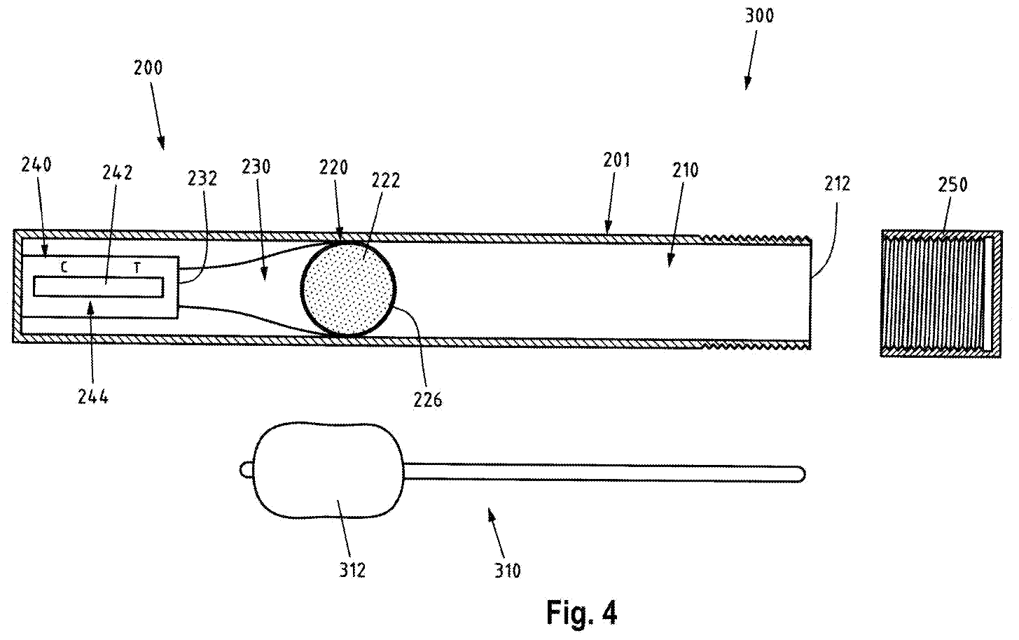

The test device according to the invention is described below by way of example with reference to FIG. 2 and FIG. 4. FIG. 2 illustrates a schematic embodiment of a test kit 300 according to the invention with a first exemplary embodiment of a test device 200 according to the invention and a sample collection stick 310 adapted to the test device 200. Furthermore, FIG. 4 illustrates a schematic embodiment of a test kit 300 according to the invention with a second exemplary embodiment of a test device 200 according to the invention and a sample collection stick 310 adapted to the test device 200.

In the embodiments of the test device 200 shown in FIG. 2 and FIG. 4, respectively, the test device 200 comprises a housing 201, wherein the housing 201 comprises:

a receiving area 210 for receiving a sample collection stick 310;

a fluid reservoir 220 forming a separated area within the housing 201 and filled with a reaction solution 222; and

an extraction area 230; and

an analysis area 240.

In the embodiments of the test device 200 shown in FIG. 2 and FIG. 4, respectively, the fluid reservoir 220 is delimited by at least one separation layer 224, 225, 226 of the fluid reservoir 220. Thereby, the at least one separation layer 224, 225, 226 is at least one membrane 224, 225, 226. By providing separation layers 224, 225, 226, in particular membranes 224, 225, 226, both the application safety of the test device 200 and the flexibility in performing the test are increased.

In the embodiment of the test device 200 shown in FIG. 2, the liquid reservoir 220 is delimited by a first separation layer 224 and a second separation layer 225 of the liquid reservoir 220, which together with a delimitation section 228 of the housing 201 form a separated area within the housing 201. Here, the first separation layer 224 bounds the liquid reservoir 220 toward the receiving area 210 of the housing 201 and the second separation layer 225 bounds the liquid reservoir 220 toward the analysis area 240 of the housing 201.

In the embodiment of the test device 200 shown in FIG. 4, the liquid reservoir 220 is delimited by a single separating layer 226 that completely encloses a cavity, in this case a spherical cavity, so that a separated area is formed within the housing 201. This results in a particularly simple structure of the test device 200. The liquid reservoir can be fixed in this case by an optional holding member (not shown).

In the embodiments of the test device 200 shown in FIG. 2 and FIG. 4, respectively, the separation layers 224, 225, 226 of the liquid reservoir 220 are arranged for this purpose to be penetrated by a sample collection stick 310 inserted into the housing 201 and pressed against the separation layers 224, 225, 226, so that a sample located on the sample collection stick 310 comes into contact with the reaction solution 222. The separation layers 224, 225, 226 can comprise predetermined penetration points (not shown) for this purpose. In this way, the tests can be performed easily and reliably, even by medically untrained personnel.

In the embodiments of the test device 200 shown in FIG. 2 and FIG. 4, respectively, the extraction area 230 is conically shaped and comprises an opening 232. This enables a process-safe supply of the sample solution to the analysis area, as well as precise dosage of which amount of sample solution is supplied to the analysis area.

In the embodiments of the test device 200 shown in FIG. 2 and FIG. 4, respectively, the analysis area 240 comprises an analysis element 242 for analyzing a sample solution. Here, the analysis element 242 is illustrated as a test strip 242 based on the lateral flow principle. However, an electronic or chip-based analysis element or any other analysis element known to the skilled person would be equally conceivable. The analysis element 242 serves to detect whether or not a corresponding organic structure is present in a sample solution.

In the embodiments of the test device 200 shown in FIG. 2 and FIG. 4, respectively, the analysis area 242 comprises a window 244 for reading a test result of the analysis element 242.

In the embodiments of the test device 200 shown in FIG. 2 and FIG. 4, respectively, the receiving area 210, the liquid reservoir 220, the extraction area 230, and the analysis area 240 are arranged one behind the other in a longitudinal direction of the housing 201. On the one hand, this results in a particularly simple structure of the test device according to the invention. On the other hand, the test device hereby enables a method according to the invention to be performed with substantially identical direction by means of a continuous movement of the sample collection stick 310. This also improves the manageability of the test device.

In the embodiments of the test device 200 shown in FIG. 2 and FIG. 4, respectively, the test device 200 further comprises a cover 250 that can be applied to the opening 212 of the receiving area 210. The illustrated locking mechanism of the cover 250 is here a screw mechanism, wherein a click mechanism would be equally conceivable. The screw mechanism shown here enables the respective compressive forces with which the moving and pressing of the sample collection stick into and against the liquid reservoir and/or the moving and pressing of the sample collection stick into and against the extraction area is performed when carrying out a method according to the invention to be precisely adjusted, so that the application safety of the test device is improved. On the other hand, the equally conceivable click mechanism enables a quick application of the cover. Preferably, the cover 250 can be applied to the opening 212 of the extraction area 210 by means of a two-stage locking mechanism (not shown). An idle time can thereby be provided between the two stages of the two-stage closure mechanism (not shown).

In the embodiment of the test device 200 shown in FIG. 2, the cover 250 comprises an optional fixation element 252 for at least temporarily fixing a sample collection stick 310. Here, the fixation element 252 is a hollow cylinder adapted to a diameter of the sample collection stick 310 and open on one side. As illustrated in FIG. 2, the fixation element 252 receives the sample collection stick 310 to fix it. However, the illustration in FIG. 2 is to be understood only as a non-limiting example. Similarly, it is also conceivable that the cover 250 of the embodiments of the test device 200 shown in FIG. 4 comprises an optional fixation element 252 for at least temporarily fixing a sample collection stick 310, wherein the fixation element 252 receives the sample collection stick 310 to fix it. In addition, it is also conceivable that in the embodiment of the test device 200 shown in FIG. 2, the sample collection stick 310 is not received and fixed by the fixation element 252, or that the fixation element 252 is omitted altogether. The fixation element 252 prevents the sample collection stick 310 from sliding off when the sample collection stick 310 is pressed against the liquid reservoir 220 and/or the extraction area 230 by means of the cover 250.

In the embodiments of the test device 200 shown in FIG. 2 and FIG. 4, respectively, the test device 200, in particular the housing 201, is a hand-held device. The housing 201 thereby comprises the shape of a tube or pipe. In this way, the test device 200 is characterized by particularly good manageability compared to the prior art.

In the embodiments of the test kit 300 shown in FIG. 2 and FIG. 4, respectively, the test kit 300 comprises, adjacent to the test device 200, a sample collection stick 310 adapted to the test device 200, in particular to a length of the housing 201. The sample collection stick is adapted to a length of the housing 201 in that the sample collection stick 310 presses against a wall of the extraction area 230 of the housing 201 when the cover 250 is applied to the opening 212 of the receiving area 210 when performing a method according to the invention. This has the effect that the method according to the invention can be performed with the test kit 300 in a particularly application-safe manner.

In the embodiments of the test kit 300 shown in FIG. 2 and FIG. 4, respectively, the test kit 300 is in three parts and comprises a housing 201, a cover 250, and a sample collection stick 310, making it particularly easy to handle compared to the prior art. As exemplarily and non-limitingly illustrated in FIG. 2, the cover 250 can also comprise an optional fixation element 252 for at least temporary or permanent fixation of the sample collection stick 310. In this way, the cover 250 and the sample collection stick 310 can also be at least temporarily firmly connected to one another to form a unit such that, together with the housing 201, a two-part test kit is obtained.

In the embodiments of the test kit 300 shown in FIG. 2 and FIG. 4, respectively, the sample collection stick 310 comprises a compressible, open-pored sample receiving area 312. Illustrated here is a sponge; however, a cotton ball would also be conceivable as a sample receiving area 312. Preferably, the sample receiving area 312 is designed as a flocked head.

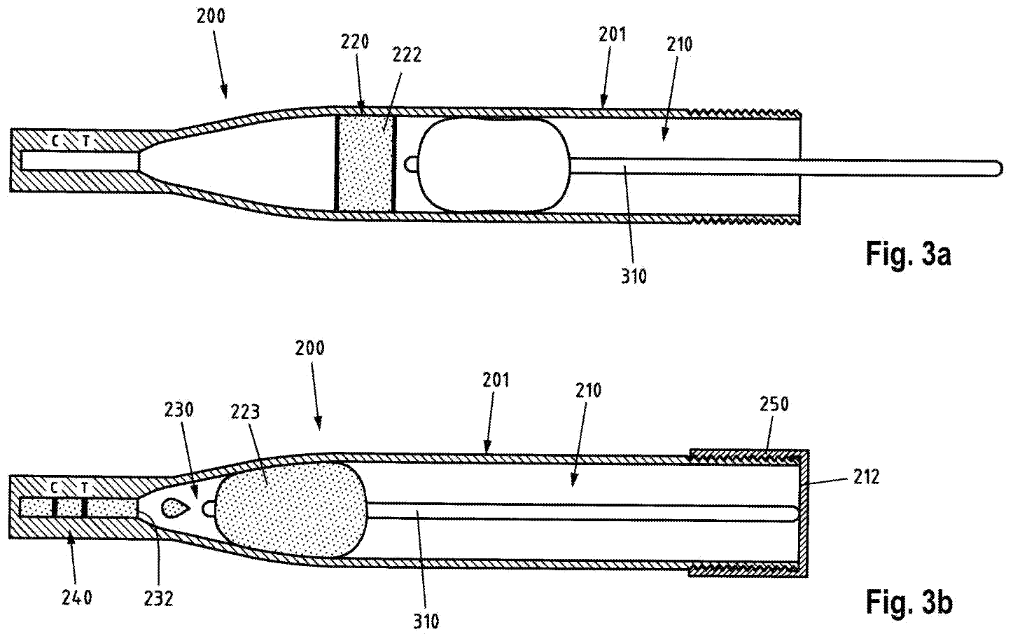

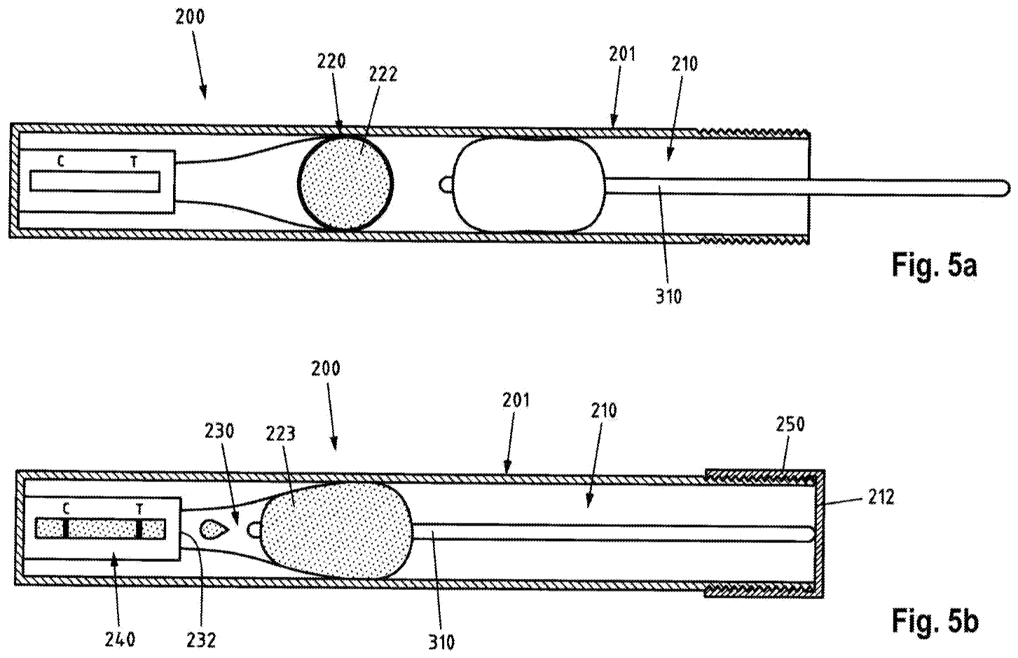

The carrying out of a method according to the invention using a test kit according to the invention is described below by way of example using FIGS. 3a, 3b, 5a and 5b. For this purpose, FIG. 3a shows the test kit from FIG. 2 in an arrangement which corresponds to a step of the method according to the invention, and FIG. 5a shows analogously the test kit from FIG. 4 in an arrangement which corresponds to the same step of the method according to the invention. Furthermore, FIG. 3b shows the test kit from FIG. 2 in an arrangement corresponding to a further step of the method according to the invention, and FIG. 5b shows analogously the test kit from FIG. 4 in an arrangement corresponding to the same step of the method according to the invention.

FIGS. 3a and 5a each show the test device 200 in an arrangement after the method step of inserting 110 the sample collection stick 310. Accordingly, the sample collection stick 310 is located in the receiving area 210 of the housing 201. A sample is located on the sample collection stick 310. By inserting 110 the sample collection stick 310 into the receiving area 210 of the housing 201, the sample is provided for performing the test within the housing 201. As illustrated in FIG. 3a in an exemplary and non-limiting manner, the sample collection stick 310 is fixed in place by an optional fixation element 252 of the cover 250 during insertion 110 into the receiving area 210. Likewise, it is also conceivable that the cover 250 of the embodiments of the test device 200 shown in FIG. 5a comprises an optional fixation element 252 for at least temporarily fixing the sample collection stick 310. In addition, it is also conceivable that in the embodiment of the test device 200 shown in FIG. 3a, the sample collection stick 310 is not received and fixed by the fixation element 252 of the cover 250 when it is inserted 110 into the receiving area, or that the fixation element 252 is omitted altogether.

In a process step not specifically shown, moving and pushing 120 the sample collection stick 310 into and against a fluid reservoir 220 that forms a separated area within the housing 201 and is filled with a reaction solution 222. The sample thereby comes into contact with the reaction solution 222, resulting in a sample solution suitable for analysis in the analysis area of the housing.

FIGS. 3b and 5b respectively show the test device 200 in an arrangement after the method step of moving and pushing 130 the sample collection stick 310 into and against the extraction region 230 of the housing 201. Here, the moving and pushing 130 is performed by placing the cover 250 on the opening 212 of the receiving area 210. Thereby, at least a portion of the sample solution 223 enters the analysis area 240 of the housing 201 through the opening 232 of the conical extraction area 230. This enables the analysis of the sample solution 223 in the analysis area 240 and thereby the performance of the test. As illustrated in FIG. 3b by way of example and not limitation, when the sample collection stick 310 is pressed 130 against the extraction area 230, it is fixed in place by an optional fixation element 252 of the cover 250. Likewise, it is also conceivable that the cover 250 of the embodiments of the test device 200 shown in FIG. 5b comprises an optional fixation element 252 for at least temporarily fixing the sample collection stick 310. In addition, it is also conceivable that in the embodiment of the test device 200 shown in FIG. 3b, the sample collection stick 310 is not fixed by the fixation element 252 of the cover 250 when pressed 130 against the extraction area 230, or that the fixation element 252 is omitted altogether.

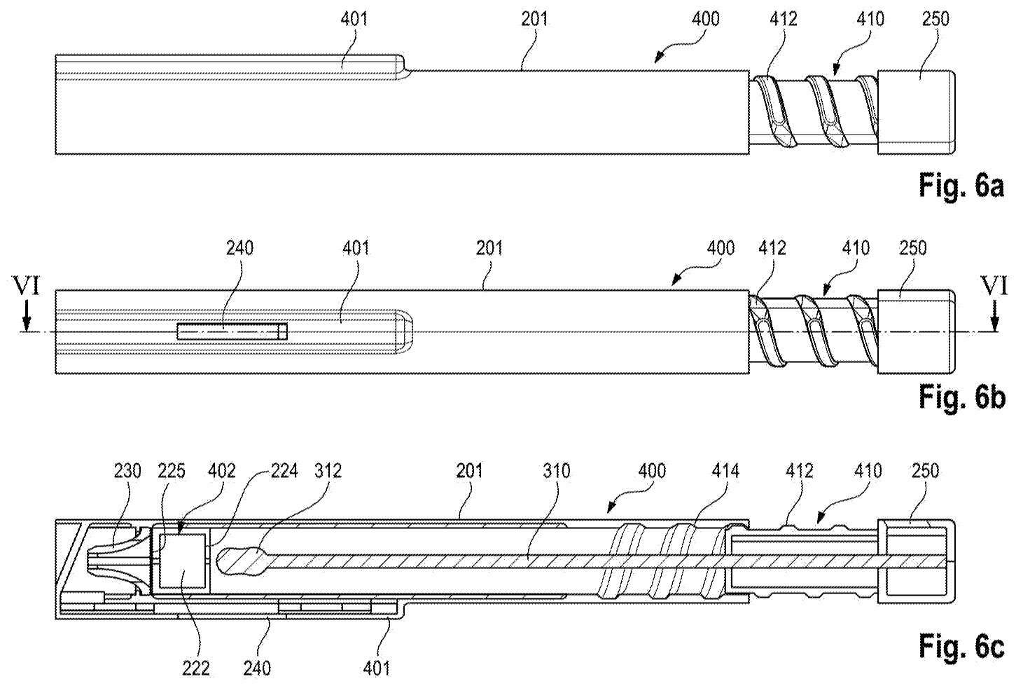

With reference to FIGS. 6a-c, 7a-c, and 8-12, further embodiments of a test device according to the invention will be explained furthermore. Thereby, only the respective test device is provided with a new reference sign, while corresponding reference numerals are used for the other corresponding parts as before.

These embodiments differ from the previously described embodiments in particular in that the analysis area 240 is not received in an extension of the housing 201, but in a lateral area 401 of the housing 201, and in that the reaction solution 222 together with the associated separating layers 224, 225 is received in a cartridge 402 which the housing 201 is insertable.

FIG. 6a shows a longitudinal view of a test device 400 according to the invention. FIG. 6b shows the test device 400 in a longitudinal view rotated by 90° with respect to FIG. 6a.

FIG. 6c shows a longitudinal section through the test device 400 along the line VI-VI shown in FIG. 6b.

The test device 400 comprises an internal thread 414 on the housing 201. A cylindrical extension 410 is formed on the cover 250, and an external thread 412 is provided on the extension 410. The external thread 412 engages an associated internal thread 414 on the housing 400. The sample collection stick 310 is fixed to the cover 250, and is mounted in the position shown in FIG. 6a-c, it is held in the as-delivered position prior to use of the test, still with a distance before the first separating layer 224.

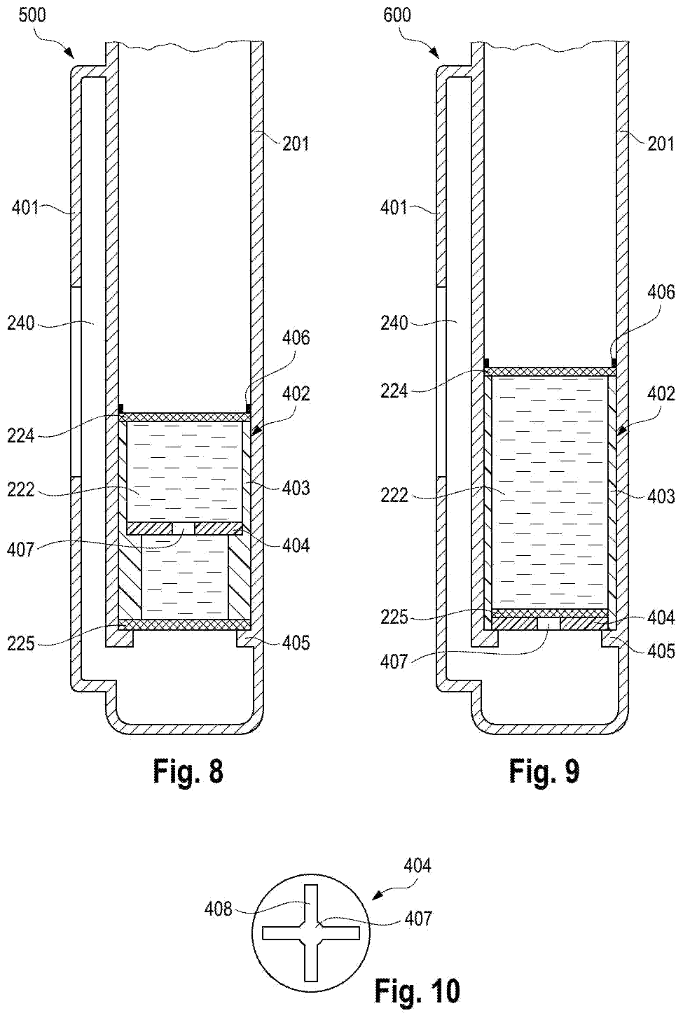

In the embodiment according to FIGS. 6a-c and 7a-c, and FIGS. 8-12, the reaction solution 222 is received in a cartridge 402 held within the housing 201. As can be seen in more detail from FIGS. 8-12, the cartridge 402 comprises a substantially cylindrical cartridge housing 403 within which the reaction solution 222 is enclosed and sealed at each end by a separating layer 224, 225 in the form of a membrane. The cartridge 402 is manufactured as a separate part and can be inserted, for example, when the housing 201 is assembled from two housing halves. Thereby, the cartridge 402 is retained at the outer end by a protrusion 405. Additional securing in the form of a bead 406 (FIG. 8 and FIG. 9) can be provided at the inner end of the cartridge 402 to prevent the cartridge 402 from slipping. After insertion of the cartridge 402, the two halves of the housing are generally connected to each other by a material bond, e.g. by heat sealing.

Providing the reaction solution 222 within a cartridge 402 that is inserted into the housing 201 has the advantage of completely decoupling the process of filling the liquid reservoir with the reaction liquid from the rest of the manufacturing process. In this way, the manufacturing process is simplified and the reaction solution can be inserted into the test device in the required amount and concentration without risk of contamination.

Furthermore, the receptacle of the analysis area 240 in a lateral area 401 of the housing 201 comprises the advantage of significantly shortening the overall length of the test device 400 compared to an arrangement according to FIGS. 2 to 4.

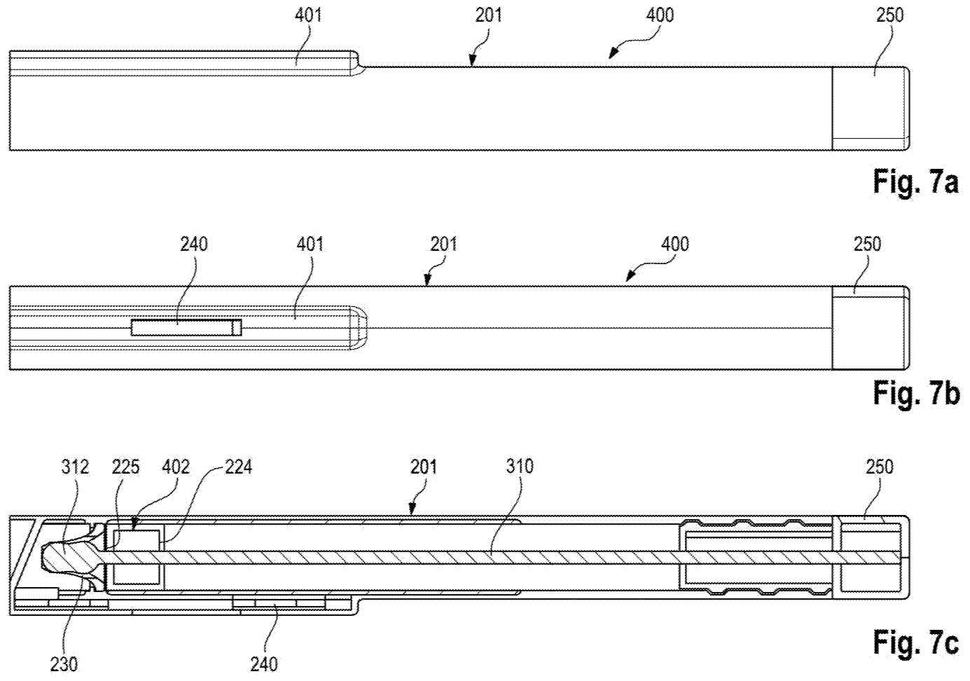

The entire test device 400 is received in a sterile package in the position shown in FIGS. 6a-c. To use the test, the sterile packaging is first removed and then the cover 250 with the sample collection stick 310 received thereon is unscrewed from the position shown in FIGS. 6a-c. The sample is then taken and the sample collection stick 310 is reinserted into the housing opening and screwed into the housing 201 by rotating the cover 250 by means of the threaded connection 412, 414. The sample collection stick 310 then first ruptures the first release layer 224 on the cartridge 402 so that the sample comes into contact with the reaction solution. After a predetermined time, the sample collection stick 310 is further screwed into the housing 201 so that the position according to FIGS. 7a-c is reached. The sample collection stick 310 engages with sample receiving area 312 in the funnel-shaped extraction area adjacent to the cartridge. As a result, the sample solution is squeezed out and transported towards the analysis area 240.

In the embodiment of the test device 500 shown in FIG. 8, a stripping element 404 is still provided within the cartridge 402 to assist in stripping the sample material from the sample collection area 312 of the sample collection stick 310. This facilitates a stripping of the sample from the stripping element 404. The stripping element 404 is preferably made of a flexible material.

The stripping element 404 can have approximately the shape shown in FIG. 10, with a circular passage 407 in the center from which openings 408 extend outwardly in approximately a cross-slot shape.

In the embodiment of the test device shown in FIG. 9 and designated 600 overall, the stripping element 404 is located at the outer end of the cartridge 402, adjacent to which is the separating element 225 in the form of the membrane.

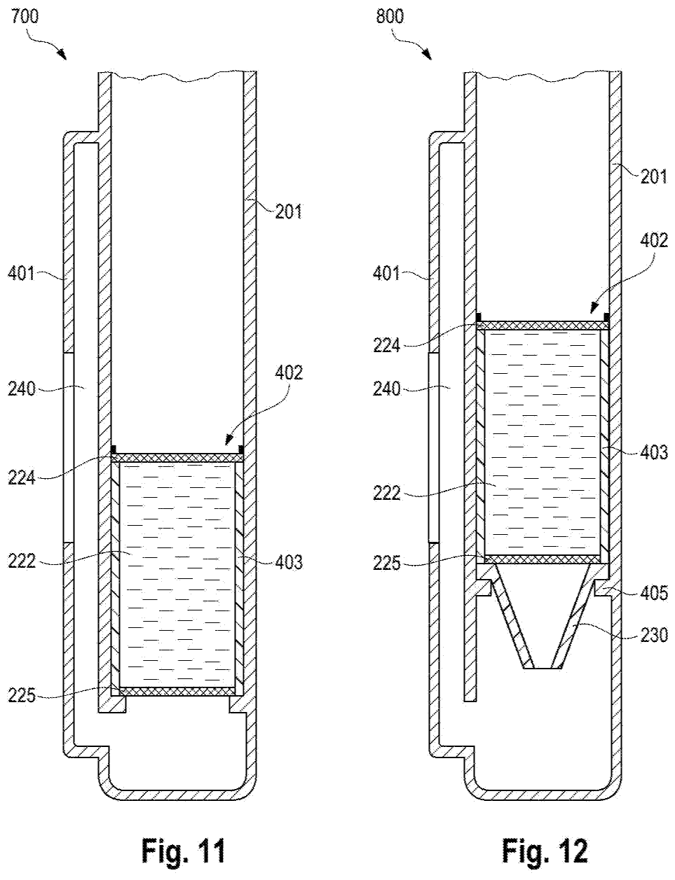

In the embodiment of the test device designated 700 in total according to FIG. 11, a stripping element and also an extraction area were dispensed with, but only the cartridge 402 with the reaction solution 222 was inserted into the housing 201. Here, the sample solution is transported into the analysis area passively by gravity and the opening of the membrane 225.

In the embodiment of the test device shown in FIG. 12, denoted overall as 800, a stripping element was omitted. Instead, a funnel-shaped extraction area 230 was inserted into the housing 201 adjacent to the separation layer 225.

A fabrication of a test device according to any of the embodiments shown in FIGS. 6a-c, 7a-c, or 8 to 12 can be performed in approximately the following ways:

The housing (201) is assembled in longitudinal direction from two housing elements. These each form a half shell. In FIG. 6c, for example, a first housing element could be the half of the housing 201 shown, while the second housing element could be configured to be complementary for this purpose.

First, the first and second housing elements are made of plastic (e.g., polyethylene), such as by injection molding. Further, the associated cover (250) is likewise made of plastic (e.g., polyethylene), such as by injection molding, and the sample collection stick 310 is fixed in the cover 250 and the cylindrical projection 410 provided thereon.

A separately manufactured cartridge 402 and an analysis element 240, such as a test strip, are inserted into the first housing element in the areas provided for this purpose. Then, the second housing element is placed on the first housing element and connected to the first housing element in a material-locking manner, for example by ultrasonic welding or heat sealing.

Subsequently, it is only necessary to screw the cover 250 with the sample collection stick 310 provided thereon into the housing 201 until the position shown in FIGS. 6a-c is reached, in which the first separation layer 224 of the cartridge 402 is still intact.

What is claimed is:

1. A test device for carrying out a test directed to the detection of an organic structure, with a housing comprising:
a receiving area for receiving a sample collection stick;
a liquid reservoir forming a separated area within the housing and filled with a reaction solution; and
an analysis area integrated into the housing.

2. The test device according to claim 1, wherein the housing is configured as a one-piece component.

3. The test device according to claim 1, wherein an extraction area is further provided in the housing.

4. The test device according to claim 1, wherein a plurality of liquid reservoirs is provided in the housing with reaction solutions received therein, each of which is delimited from one another and from the outside by separation layers.

5. The test device according to claim 1, wherein a cartridge containing at least one liquid reservoir is received in the housing, wherein each liquid reservoir is delimited by membranes.

6. The test device according to claim 5, in which the cartridge is fixed in the housing by means of a clamp or click connection.

7. The test device according to claim 2, wherein an extraction area is provided at the outer end of the cartridge.

8. The test device according to claim 2, wherein an extraction area is tapered in a funnel shape.

9. The test device according to claim 5, wherein the cartridge comprises a stripping element for stripping the sample collection stick.

10. The test device according to claim 1, wherein the analysis area comprises:
an analysis element for analyzing a sample solution; and
a window for reading a test result of the analysis element.

11. The test device of claim 10, wherein the analysis element is a test strip (242) according to the lateral flow principle.

12. The test device of claim 10, wherein the analysis element is an electronic or chip-based analysis element.

13. The test device according to claim 1, wherein the receiving area, the fluid reservoir, the analysis area, and, if present, an extraction area are arranged one behind the other in a longitudinal direction of the housing.

14. The test device according to claim 1, wherein the housing comprises a laterally outwardly projecting area in which the analysis element is received.

15. The test device according to claim 14, wherein the analysis element is connected via a feedthrough to an area adjacent to a separation layer of the fluid reservoir or, if present, to the exit of an extraction area via a cavity.

16. The test device according to claim 1, wherein a cover is provided on an opening of the housing, which cover is movable on the housing by means of a two-stage or multi-stage locking mechanism.

17. The test device according to claim 1, wherein a cover is provided on an opening of the housing, which cover is movable on the housing by means of a two-stage click mechanism or by means of a two-stage screw mechanism or by means of a two-stage combined click-screw or screw-click mechanism.

18. The test device according to claim 16, wherein the sample collection stick is retained in the housing and secured by the cover.

19. The test device according to claim 16, wherein the sample collection stick is movable by movement of the cover into the housing against a first separation layer of the liquid reservoir for opening thereof, and is movable against any further separation layer for release of the liquid from the liquid reservoir or a further liquid reservoir towards the analysis area.

20. A test device according to claim 5, wherein the housing is composed of at least two housing elements that are sealingly connected to each other by a material connection, wherein the analysis element and the cartridge are fixed between the housing elements at their intended position.

21. The test device according to claim 20, wherein further an extraction area or a stripping element is received between the two housing elements.

22. A method for manufacturing a test device for carrying out a test directed to the detection of an organic structure, said test device comprising a housing consisting of plastic, which comprises a receiving area for receiving a sample collection stick, as well as a liquid reservoir which comprises a separated area within the housing and is filled with a reaction solution, and an analysis area integrated into the housing, wherein the housing is composed of at least two housing elements in the longitudinal direction, said method comprises the following steps:
manufacturing a first housing element;
manufacturing a second housing element;
providing a sample collection stick;
manufacturing a cover of plastic material longitudinally fixable to the housing with a screw or click connection; and fixing the sample collection stick to the cover;
manufacturing a cartridge containing at least one liquid reservoir, wherein each liquid reservoir is delimited by separation layers;
providing an analysis element;
wherein the housing is divided in longitudinal direction into at least the first and the second housing element such that the cartridge and the analysis element can be inserted into areas of the first housing element provided for this purpose;
inserting the cartridge and the analysis element into the first housing element;
placing the second housing element onto the first housing element;
materially connecting the first and second housing elements to form the housing; and
placing the cover with the sample collection stick held thereon on the screw or click connection of the housing and inserting the sample collection stick into a cavity of the housing to such an extent that an end of the sample collection stick intended for sampling is held on the cartridge at a distance from a first separation layer.

23. The method of claim 22, wherein the test device is the test device according to claim 1.

24. The method according to claim 22, in which the material connection is made by ultrasonic welding, heat sealing or bonding.

25. A method for carrying out a test directed to the detection of an organic structure comprising the following steps:
inserting a sample collection stick, on which at least one sample is located, into a receiving area of a housing of a test device;
moving the sample collection stick into a liquid reservoir that forms a separate area within the housing and is filled with a reaction solution so that the at least one sample contacts the reaction solution, thereby forming a sample solution; and
moving the sample collection stick into the housing so that at least a portion of the sample solution enters an analysis area of the housing.

26. The method of claim 25, wherein the test device is the test device according to claim 1.
